(19) United States
(12) Patent Application Publication
(10) Pub. No.: US 2019/0282655 A1
(43) Pub. Date: Sep. 19, 2019

(54) Treatment of Constipation-Predominant Irritable Bowel Syndrome

(71) Applicants: Ironwood Pharmaceuticals, Inc., Cambridge, MA (US); Forest Laboratories Holdings Limited, Hamilton (BM)

(72) Inventors: Jeffrey Johnston, Boston, MA (US); Bernard Joseph Lavins, Lexington, MA (US); Harvey Schneier, New York, NY (US)

(21) Appl. No.: 16/414,946

(22) Filed: May 17, 2019

Related U.S. Application Data
(63) Continuation of application No. 16/136,523, filed on Sep. 20, 2018, now abandoned.
Application No. 16/136,523 is a continuation of application No. 15/879,670, filed on Jan. 25, 2018, now abandoned.
Application No. 15/879,670 is a continuation of application No. 15/624,862, filed on Jun. 16, 2017, now abandoned.
Application No. 15/624,862 is a continuation of application No. 15/350,799, filed on Nov. 14, 2016, now abandoned.
Application No. 15/350,799 is a continuation of application No. 15/086,483, filed on Mar. 31, 2016, now abandoned.
Application No. 15/086,483 is a continuation of application No. 14/826,478, filed on Aug. 14, 2015, now abandoned.
Application No. 14/826,478 is a continuation of application No. 14/580,500, filed on Dec. 23, 2014, now abandoned.
Application No. 14/580,500 is a continuation of application No. 13/821,761, filed on Feb. 14, 2014, now abandoned.
(60) Provisional application No. 61/408,994, filed on Nov. 1, 2010.
(60) Provisional application No. 61/408,509, filed on Oct. 29, 2010.
(60) Provisional application No. 61/394,267, filed on Oct. 18, 2010.
(60) Provisional application No. 61/382,469, filed on Sep. 13, 2010.
(60) Provisional application No. 61/381,936, filed on Sep. 11, 2010.

(51) Int. Cl. A61K 38/12; A61K 9/48; A61K 9/16; A61K 38/10; A61K 31/198

(52) U.S. Cl. CPC A61K 38/12; A61K 9/485; A61K 9/4866; A61K 9/4858; A61K 2300/00; A61K 38/10; A61K 31/198; A61K 9/4808; A61K 9/1676

(57) ABSTRACT

The invention provides methods for treating a patient with constipation-predominant irritable bowel syndrome by administering a therapeutically effective dose of linaclotide.

### PRIORITY CLAIM

This application is a continuation of, and claims priority to U.S. patent application Ser. No. 16/136,523 filed Sep. 20, 2018, which is a continuation of U.S. patent application Ser. No. 15/879,670 filed Jan. 25, 2018, which is a continuation of U.S. patent application Ser. No. 15/624,862 filed Jun. 16, 2017, which is a continuation of U.S. patent application Ser. No. 15/350,799 filed Nov. 14, 2016, which is a continuation of U.S. patent application Ser. No. 15/086,483 filed Mar. 31, 2016, which is a continuation of U.S. patent application Ser. No. 14/826,478 filed Aug. 14, 2015, which is a continuation of U.S. patent application Ser. No. 14/580,500 filed Dec. 23, 2014, which is a continuation of U.S. patent application Ser. No. 13/821,761 filed Mar. 8, 2013, which is the United States National Phase filing of PCT/US2011/051080 filed Sep. 9, 2011. This application also claims priority to U.S. Provisional Patent Application Ser. No. 61/381,936, filed Sep. 11, 2010; U.S. Provisional Patent Application Ser. No. 61/382,469, filed Sep. 13, 2010; U.S. Provisional Patent Application Ser. No. 61/394,267, filed Oct. 18, 2010; U.S. Provisional Patent Application Ser. No. 61/408,509, filed Oct. 29, 2010, and U.S. Provisional Patent Application Ser. No. 61/408,994, filed Nov. 1, 2010. The entire contents of the aforementioned applications are incorporated herein by reference.

### FIELD OF THE INVENTION

This invention relates to the use of linaclotide to treat constipation-predominant irritable bowel syndrome.

### SEQUENCE LISTING

This application incorporates by reference in its entirety the Sequence Listing entitled “IW082PCT1US1CON1_ST25.txt” (821 bytes) which was and last modified on Dec. 22, 2014 and filed electronically herewith.

### BACKGROUND

As many as 11 million Americans suffer from symptoms associated with constipation-predominant irritable bowel syndrome (IBS-C). IBS-C is a chronic functional gastrointestinal disorder characterized by abdominal pain, discomfort, and bloating associated with altered bowel habits. There are currently few available therapies to treat this disorder and there is a high rate of dissatisfaction with them. Patients suffering from IBS-C can be affected physically, psychologically, socially, and economically. IBS-C significantly affects patients' quality of life by impairing their ability to work and participate in typical daily activities.

Rome II Diagnostic Criteria for irritable bowel syndrome (IBS) includes at least 12 weeks (which need not be consecutive) within the preceding 12 months of abdominal discomfort or pain with two of the following features:

(a) The abdominal discomfort or pain is relieved with defecation;

(b) Onset of symptoms is associated with a change in frequency of stools;

(c) Onset of symptoms is associated with a change in the form (appearance) of stool.

Constipation-predominant irritable bowel syndrome in a patient may be further defined as the presence of fewer than three bowel movements (BMs) per week and by one or more of the following symptoms for at least 12 weeks, which need not be consecutive:

(a) Straining during greater than 25% of BMs;

(b) Lumpy or hard stool during greater than 25% of BMs; and

(c) Sensation of incomplete evacuation during greater than 25% of BMs (see, e.g., Rome II criteria (Drossman, 2000), slightly modified from the original).

Patients with IBS-C may also report symptoms that include (i) alternation between constipation and normal stools, and (ii) lower abdominal cramping, aching or discomfort that is commonly triggered by eating.

Currently, only one FDA-approved therapy for the treatment of IBS-C is available. Therefore, there remains an unmet medical need for additional, well-tolerated, and effective therapies for patients with IBS-C that not only increase bowel frequency but also relieve other various associated symptoms including abdominal pain and discomfort.

### SUMMARY

In general, the invention relates to a method of treating irritable bowel syndrome associated with constipation.

In one aspect, the method of treating a patient with constipation-predominant irritable bowel syndrome includes administering a therapeutically effective dose of linaclotide.

In certain embodiments, the method of treating a patient with constipation-predominant irritable bowel syndrome includes administering a therapeutically effective dose of linaclotide once a day. In other embodiments, the therapeutically effective dose is administered once a day in the morning.

In one aspect, the method of treating a patient with constipation-predominant irritable bowel syndrome includes administering a therapeutically effective dose of linaclotide once a day at least 30 minutes before ingestion of food. In other aspects, the therapeutically effective dose is administered at least 30 minutes before breakfast. In other aspects, the therapeutically effective dose is administered at least 120 minutes before ingestion of food.

In aspects of the present invention, the therapeutically effective dose is 100 to 600 μg linaclotide (e.g., 266 μg to 300 μg linaclotide, 266 μg linaclotide). In some aspects, the linaclotide is in the form of a tablet or capsule.

In some aspects, the linaclotide is administered for a period of greater than four weeks, (e.g., at least 12 weeks; at least 26 weeks). In aspects of the present method, the linaclotide is administered each day of the week, at least once a week, at least twice a week, at least three times a week, at least four times a week, at least five times a week or at least six times a week.

In one aspect, the method of treating a patient with constipation-predominant irritable bowel syndrome includes administering a therapeutically effective dose of linaclotide, wherein the administering decreases abdominal pain in said patient compared to said patient prior to treatment with linaclotide.

In another aspect, the method of treating a patient with constipation-predominant irritable bowel syndrome includes administering a therapeutically effective dose of linaclotide, wherein the administering increases the number of complete spontaneous bowel movements (CSBMs) by the patient to three or greater CSBMs per week. In some aspects, the administering increases the spontaneous bowel movement (SBM) frequency rate in said patient compared to said patient prior to treatment with linaclotide. In some aspects, the CSBMs are increased by at least one per week in treated patients, compared to prior to treatment with linaclotide.

In another aspect, the method of treating a patient with constipation-predominant irritable bowel syndrome includes administering a therapeutically effective dose of linaclotide, wherein the administering decreases abdominal pain in said patient compared to said patient prior to treatment with linaclotide and increases the number of complete spontaneous bowel movements (CSBMs) by the patient to three or greater CSBMs per week.

In some aspects of the present methods, administering of linaclotide provides sustained relief from symptoms of constipation predominant irritable bowel syndrome, sustained relief from symptoms of constipation predominant irritable bowel syndrome for at least 16 weeks, sustained relief from symptoms of constipation predominant irritable bowel syndrome for at least 26 weeks, sustained relief from symptoms of constipation predominant irritable bowel syndrome for at least 1 out of 2 weeks, sustained relief of symptoms of constipation predominant irritable bowel syndrome for at least 3 out of 4 weeks, 6 out of 12 weeks, or 9 out of 16 weeks.

In another aspect, the method of treating a patient with constipation-predominant irritable bowel syndrome includes administering a therapeutically effective dose of linaclotide, wherein the administering improves abdominal symptoms (e.g., pain, discomfort, bloating) and bowel symptoms (e.g., CSBMs/per week, SBMs/per week, stool consistency, and straining) in a patient with greater than moderate abdominal pain, in a patient with moderate to severe abdominal pain, or in a patient with severe to very severe abdominal pain.

In certain embodiments of the foregoing aspects, the methods include administering linaclotide formulation including:

(a) linaclotide or pharmaceutically acceptable salts thereof;

(b) CaCl₂;

(c) L-Leucine; and

(d) Hydroxypropyl Methylcellulose

wherein linaclotide is present in the formulation in an amount between 100 μg to 600 μg and the molar ratio of Ca²⁺:leucine linaclotide is between 5-100:5-50:1. In some embodiments, the formulation contains 133 μg of linaclotide. In some embodiments, the formulation contains 266 μg of linaclotide. In some aspects, the formulation is in the form of a tablet or capsule.

In some aspects, discontinuing the administration of a therapeutically effective dose of linaclotide does not produce a rebound of the symptoms in said patient. In some aspects, at least one of the IBS-C symptoms does not rebound, wherein the symptoms are selected from: a decrease in the number of CSBMs per week; a decrease in the number of SBMs per week; an increase in bloating, an increase in abdominal discomfort; an increase in abdominal pain; an increase in constipation severity; a decrease in stool consistency; or an increase in straining during defecation.

In one aspect, methods of optimizing the treatment with linaclotide of a patient with constipation-predominant irritable bowel syndrome are provided, comprising administering a first therapeutically effective dose of linaclotide once a day; determining whether the patient develops loose stools or diarrhea after treatment with linaclotide; wherein if the patient develops loose stools or diarrhea after one or more days of said administering, administering a second therapeutically effective dose of linaclotide once a day, wherein said second therapeutically effective dose is lower than said first therapeutically effective dose. In some aspects, the first therapeutically effective dose of linaclotide is 266 μg and the second therapeutically effective dose of linaclotide is 133 μg.

In one aspect, a method of treating a patient with constipation predominant irritable bowel syndrome is provided, comprising administering a therapeutically effective dose of a GC-C agonist and wherein discontinuing the administration of a therapeutically effective dose of a GC-C does not produce a symptom rebound of constipation predominant irritable bowel syndrome for said patient.

Importantly, administration of linaclotide as described herein provides one or more of the following advantages: an increase in the number of complete spontaneous bowel movements (CSBMs) by the patient compared to said patient prior to treatment with linaclotide; a decrease in abdominal pain or discomfort in said patient compared to said patient prior to treatment with linaclotide; a decrease in bloating, such as abdominal bloating, in said patient compared to said patient prior to treatment with linaclotide; a decrease in the abdominal discomfort in said patient compared to said patient prior to treatment with linaclotide; a decrease in the constipation severity in said patient compared to said patient prior to treatment with linaclotide; an improvement in the stool consistency in said patient compared to said patient prior to treatment with linaclotide; an improvement in the bowel movement (BM) frequency in said patient compared to said patient prior to treatment with linaclotide; a decrease in the straining during defecation in said patient compared to said patient prior to treatment with linaclotide; and an improvement in patient assessment of quality of life.

### BRIEF DESCRIPTION OF THE DRAWINGS

FIG. 1A depicts an overview of the Trial 1 (LIN-MD-31) study design; FIG. 1B depicts an overview of the Trial 2 (MCP-103-302) study design.

FIG. 2 depicts the results and improvements of the 9/12 week APC 3+1 patient responder for Trial 1 and 2.

FIG. 3 depicts the results and improvements of the 9/12 week CSBM 3+1 patient responder for Trial 1 and 2.

FIG. 4 depicts the results and improvements of the 9/12 week abdominal pain patient responder for Trial 1 and 2.

FIG. 5 depicts the results and improvements of the 6/12 week APC +1 patient responder for Trial 1 and 2.

FIG. 6 depicts the results and improvements of the 6/12 week abdominal pain patient responder for Trial 1 and 2.

FIG. 7 depicts the results and improvements of the 6/12 week CSBM +1 patient responder for Trial 1 and 2.

FIG. 8 depicts the results and improvements of the 12 week abdominal pain/discomfort patient responder for Trial 1 and 2.

FIG. 9 depicts the results and improvements of the 12 week IBS degree of relief patient responder for Trial 1 and 2.

FIG. 10 depicts the distribution of 12 week abdominal pain improvement responses for Trial 1 (LIN-MD-31).

FIG. 11A depicts the reduction of abdominal pain during the first week of treatment in Trial 1, which is sustained throughout the trial.

FIG. 11B depicts the reduction of abdominal pain during the first week of treatment in Trial 2, which is sustained throughout the 26 weeks of treatment.

FIG. 11C depicts the 12-week mean reduction in abdominal pain from baseline in Trial 1 and 2.

FIG. 12A depicts the improvement of CSBMs during the first week of treatment in Trial 1, which is sustained throughout the trial.

FIG. 12B depicts the improvement of CSBMs during the first week in Trial 2, which is sustained throughout 26 weeks.

FIG. 12C depicts the 12 week CSBM frequency rate for Trial 1 and Trial 2.

FIG. 13 depicts an analysis of linaclotide by HPLC.

These figures are provided by way of example and are not intended to limit the scope of the present invention.

### DETAILED DESCRIPTION

### Definitions

As used herein, the term “binder” refers to any pharmaceutically acceptable binder that may be used in the practice of the invention. Examples of pharmaceutically acceptable binders include, without limitation, a starch (e.g., corn starch, potato starch and pre-gelatinized starch (e.g., STARCH 1500® and STARCH 1500 LM®, sold by Colorcon, Ltd. and other starches), maltodextrin, gelatin, natural and synthetic gums such as acacia, powdered tragacanth, guar gum, cellulose and its derivatives (e.g., methylcellulose, hydroxyethyl cellulose, hydroxyethyl methylcellulose, hydroxypropyl cellulose and hydroxypropyl methylcellulose (hypromellose), ethyl cellulose, cellulose acetate, carboxymethyl cellulose calcium, sodium carboxymethyl cellulose, carboxymethylcellulose, microcrystalline cellulose (e.g., AVICEL™, such as, AVICEL-PH-101™, -103™ and -105™, sold by FMC Corporation, Marcus Hook, Pa., USA), polyvinyl alcohol, polyvinyl pyrrolidone (e.g., polyvinyl pyrrolidone K30), and mixtures thereof.

As used herein, the term “filler” refers to any pharmaceutically acceptable filler that may be used in the practice of the invention. Examples of pharmaceutically acceptable fillers include, without limitation, talc, calcium carbonate (e.g., granules or powder), dibasic calcium phosphate, tribasic calcium phosphate, calcium sulfate (e.g., granules or powder), microcrystalline cellulose (e.g., Avicel PH101 or Celphere CP-305), powdered cellulose, dextrates, kaolin, mannitol, silicic acid, sorbitol, starch (e.g., Starch 1500), pre-gelatinized starch, lactose, glucose, fructose, galactose, trehalose, sucrose, maltose, isomalt, raffinose, maltitol, melezitose, stachyose, lactitol, palatinite, xylitol, myoinositol, and mixtures thereof.

Examples of pharmaceutically acceptable fillers that may be particularly used for coating with linaclotide include, without limitation, talc, microcrystalline cellulose (e.g., Avicel PH101 or Celphere CP-305), powdered cellulose, dextrates, kaolin, mannitol, silicic acid, sorbitol, starch, pre-gelatinized starch, lactose, glucose, fructose, galactose, trehalose, sucrose, maltose, isomalt, dibasic calcium phosphate, raffinose, maltitol, melezitose, stachyose, lactitol, palatinite, xylitol, mannitol, myoinositol, and mixtures thereof.

As used herein, the term “additives” refers to any pharmaceutically acceptable additive. Pharmaceutically acceptable additives include, without limitation, disintegrants, dispersing additives, lubricants, glidants, antioxidants, coating additives, diluents, surfactants, flavoring additives, humectants, absorption promoting additives, controlled release additives, anti-caking additives, anti-microbial agents (e.g., preservatives), colorants, desiccants, plasticizers and dyes.

As used herein, an “excipient” is any pharmaceutically acceptable additive, filler, binder or agent.

As used herein, “spontaneous bowel movement” or SBM, is a bowel movement that occurs in the absence of laxative, enema, or suppository usage within the preceding 24 hours.

As used herein, a “complete spontaneous bowel movement” or CSBM is an SBM that is accompanied by the patient self-reporting a feeling of complete emptying of the bowel.

As used herein, a “CSBM weekly responder” is a patient who had three or more CSBMs per week and an increase of at least one CSBM per week over baseline.

As used herein, a “12-week CSBM overall responder” is a patient who is a CSBM weekly responder for at least nine of the 12 weeks of the treatment period.

As used herein, “Bristol Stool Form Scale” or BSFS is seven-point scale measuring stool consistency. BSFS is a surrogate marker of gastrointestinal transit time.

As used herein, “Abdominal Pain Responder” is a patient who has a decrease of ≥30% in the mean abdominal pain score from baseline that week.

As used herein, “severity of symptoms” are self-rated or assessed by a patient, or by a medical professional on the basis of described or assessed symptoms and may be expressed on a relative scale with one or more severity categories. A “change in severity” is a rating of severity of perceived or assessed symptoms at least one scale category higher or lower than previously recorded for a patient, and corresponds to a perceived or assessed change in symptom severity (e.g., pain, discomfort, bloating). A severity scale may consist of, for example, two or more ranked or ordinal categories. An example severity scale for self-assessment of abdominal pain may include “1=none or no symptoms”, “2=mild symptoms”, “3=moderate symptoms”, “4=severe symptoms” and “5=very severe symptoms.” Two scales rating the severity of the same symptoms using a different number of categories may be made approximately equivalent to each other by combining one or more categories in one or both scales to give the same amount of categories.

As used herein, “CSBM 3+1 Responder” is a patient who has a CSBM weekly rate of ≥3 and an increase of ≥1 in the CSBM weekly rate.

As used herein, “APC 3+1 Responder Patient” is both a weekly abdominal pain responder and a weekly CSBM 3+1 responder for that week.

As used herein, “CSBM +1 Responder” is a patient who has an increase of ≥1 in CSBM weekly rate from baseline for that week.

As used herein, “APC +1 Responder Patient” is both a weekly abdominal pain responder and a weekly CSBM +1 responder for that week.

As used herein, “12-week Abdominal Pain/Abdominal Discomfort Responder” is a patient who has a decrease of ≥30% in either the mean abdominal pain score (11-point numerical rating scale) or mean abdominal discomfort score (11-point numerical rating scale) with neither score worsening from baseline for 6/12 weeks.

As used herein, “12-week IBS Degree of Relief Responder” is a patient who has an assessment of “considerably relieved” or “completely relieved” for ≥6/12 weeks.

As used herein, “rebound” is the exacerbation of the severity of a symptom experienced by a patient after discontinuation of a treatment as compared to the severity of the symptom experienced by the patient prior to that treatment.

As used herein, “rapid relief” is the improvement of one or more symptoms described herein within one week of initiating a treatment as described herein.

### Guanylate Cyclase

Guanylate cyclase C (GC-C) is a transmembrane receptor that is located on the apical surface of epithelial cells in the stomach and intestine. The receptor has an extracellular ligand-binding domain, a single transmembrane region and a C-terminal guanylyl cyclase domain. When a ligand binds to the extracellular domain of GC-C, the intracellular catalytic domain catalyzes the production of cGMP from GTP. In vivo, this increase in intracellular cGMP initiates a cascade of events that leads to increased secretion of chloride and bicarbonate into the intestinal lumen, increased luminal pH, decreased luminal sodium absorption, increased fluid secretion, and acceleration of intestinal transit. cGMP, which is secreted bidirectionally from the epithelium into the mucosa and lumen, has also been shown to dampen afferent C-fiber firing, suggesting a potential mechanism for the observed analgesic effects of GC-C agonists on visceral pain.

Linaclotide is a peptide GC-C agonist that is orally administered and currently in clinical trials for treatment of constipation-predominant irritable bowel syndrome (IBS-C) and chronic constipation (CC). In Phase 2b studies for CC, linaclotide reduced constipation, abdominal discomfort, and bloating throughout the four-week treatment period. Orally administered linaclotide acts locally by activating GC-C at the luminal surface; there are no detectable levels of linaclotide seen systemically after oral administration at therapeutic dose levels.

Linaclotide is a 14 amino acid peptide having the sequence Cys1 Cys2 Glu3 Tyr4 Cys5 Cys6 Asn7 Pro8 Ala9 Cys10 Thr11 Gly12 Cys13 Tyr14 with disulfide bonds between Cys1 and Cys6, between Cys2 and Cys10 and between Cys5 and Cys13.

The dose range of linaclotide for adult humans is generally from 25 μg to 6 mg per day orally. In a further embodiment, the dose range is 25 μg to 2 mg per day orally. In some embodiments, the dose range for adult humans is 50 μg to 1 mg per day orally (e.g., 50 μg, 67.5 μg, 100 μg, 133 μg, 150 μg, 200 μg, 250 μg, 266 μg, 300 μg, 350 μg, 400 μg, 450 μg, 500 μg, 550 μg, 600 μg, 650 μg, 700 μg, 750 μg, 800 μg, 850 μg, 900 μg, 950 μg or 1 mg). In further embodiments, the dose range is 100 μg to 600 μg per day orally. In other embodiments, the dose is 50 μg, 67.5 μg, 100 μg, 133 μg, 150 μg, 200 μg, 266 μg, 300 μg, 400 μg, 500 μg or 600 μg linaclotide per day orally.

### Administration of Peptides and GC-C Receptor Agonists

For treatment of gastrointestinal disorders, the peptides and agonists of the invention are preferably administered orally, e.g., as a tablet, gel, paste, sachet, a pellet, a capsule, a slurry, a liquid, a powder or in some other form. Orally administered compositions can include, for example, binders, lubricants, inert diluents, lubricating, surface active or dispersing additives, flavoring additives, and humectants. Orally administered formulations such as tablets may optionally be coated or scored and may be formulated so as to provide sustained, delayed or controlled release of the linaclotide therein. The linaclotide can be co-administered or co-formulated with other medications. In one embodiment, the linaclotide composition can be co-administered with other medications used to treat gastrointestinal disorders.

In certain embodiments, the linaclotide composition is provided in a unit dosage form. In some embodiments, the unit dosage form is a capsule, a tablet, a sachet, a pellet or a powder. In one such embodiment, the unit dosage form is a capsule or tablet. Such unit dosage forms may be contained in a container such as, without limitation, a paper or cardboard box, a glass or plastic bottle or jar, a re-sealable bag (for example, to hold a “refill” of tablets for placement into a different container), or a blister pack with individual doses for pressing out of the pack according to a therapeutic schedule. It is feasible that more than one container can be used together in a single package to provide a single dosage form. For example, tablets or capsules may be contained in a bottle which is in turn contained within a box. In some embodiments, the unit dosage forms are provided in a container further comprising a desiccant. In a further embodiment, the unit dosage forms, e.g., a quantity of tablets or capsules, are provided in a container, e.g., a bottle, jar or re-sealable bag, containing a desiccant.

In a further embodiment, the container containing the unit dosage forms is packaged with administration or dosage instructions. In certain embodiments, the linaclotide composition is provided in a kit. The linaclotide composition described herein and combination therapy agents can be packaged as a kit that includes single or multiple doses of two or more agents, each packaged or formulated individually, or single or multiple doses of two or more agents packaged or formulated in combination. Thus, the linaclotide composition can be present in a first container, and the kit can optionally include one or more agents in a second container. The container or containers are placed within a package, and the package can optionally include administration or dosage instructions.

In various embodiments, the unit dosage form is administered with food at any time of the day, without food at any time of the day, with food after an overnight fast (e.g., with breakfast). In various embodiments, the unit dosage form is administered once a day, twice a day or three times a day. The unit dosage form can optionally comprise other additives. In some embodiments, one, two or three unit dosage forms will contain the daily oral dose of linaclotide. The precise amount of compound administered to a patient will be the responsibility of the attendant physician. However, the dose employed will depend on a number of factors, including the age and sex of the patient, the precise disorder being treated, and its severity.

### Methods of Treating Constipation-Predominant Irritable Bowel Syndrome

In some aspects, the invention provides a method of treating a patient with constipation-predominant irritable bowel syndrome, comprising administering a therapeutically effective dose of linaclotide once a day.

In some embodiments, the therapeutically effective dose is administered once a day in the morning.

In other embodiments, the therapeutically effective dose is administered at least 30 minutes before ingestion of food.

In other embodiments, the therapeutically effective dose is administered at least 30 minutes before breakfast.

In other embodiments, the therapeutically effective dose is administered at least 120 minutes before the ingestion of food.

In still other embodiments, the therapeutically effective dose is 100 to 600 μg linaclotide. For instance, in some specific embodiments, the therapeutically effective dose is 133 g, 150 μg, 266 μg, or 300 μg of linaclotide.

In other embodiments, the therapeutically effective dose is 266 μg or 300 μg linaclotide.

In further embodiments, the therapeutically effective dose is 266 μg linaclotide.

In still other embodiments, the linaclotide is administered for a period of greater than four weeks.

In other embodiments, the linaclotide is administered for a period of at least 12 weeks.

In other embodiments, the linaclotide is administered each day of the week.

In still other embodiments, the linaclotide is administered at least once a week, at least twice a week, at least three times a week, at least four times a week, at least five times a week or at least six times a week.

In other embodiments, the linaclotide is provided in a formulation comprising
(a) linaclotide or pharmaceutically acceptable salts thereof;
(b) CaCl₂;
(c) L-Leucine; and
(d) Hydroxypropyl Methylcellulose

wherein linaclotide is present in the formulation in an amount between 100 μg to 600 μg and the molar ratio of Ca2+:leucine:linaclotide is between 5-100:5-50:1.

In still other embodiments, the formulation contains 133 μg of linaclotide.

In other embodiments, the formulation contains 266 μg of linaclotide.

In still other embodiments, the linaclotide is provided as a capsule or tablet.

In further embodiments, the linaclotide is provided as a capsule.

In still other embodiments, the administering of linaclotide decreases abdominal pain in said patient compared to said patient prior to treatment with linaclotide.

In other embodiments, the administering of linaclotide increases the number of complete spontaneous bowel movements (CSBMs) by the patient to three or greater CSBMs per week.

In other embodiments, the administering of linaclotide increases the number of CSBMs by the patient by at least one CSBM per week compared to said patient prior to treatment with linaclotide.

In some embodiments, the administering of linaclotide increases the number of CSBMs by the patient to three or greater CSBMs per week and increases the number of CSBMs by the patient by at least one CSBM per week compared to the number of CSBMs by said patient prior to treatment with linaclotide.

In some embodiments, the administering of linaclotide decreases abdominal pain in said patient compared to said patient prior to treatment with linaclotide; increases the number of CSBMs by the patient to three or greater CSBMs per week; and increases the number of CSBMs by the patient by at least one CSBM per week compared to the number of CSBMs by said patient prior to treatment with linaclotide.

In some embodiments, the administering of linaclotide increases the CSBM frequency rate in said patient compared to said patient prior to treatment with linaclotide.

In some embodiments, the administering of linaclotide increases the SBM frequency rate in said patient compared to said patient prior to treatment with linaclotide.

In some embodiments, the administering of linaclotide decreases the severity of straining during defecation in said patient compared to said patient prior to treatment with linaclotide.

In some embodiments, the administering of linaclotide decreases bloating in said patient compared to said patient prior to treatment with linaclotide.

In some further embodiments, the administering of linaclotide decreases bloating in said patient compared to said patient prior to treatment with linaclotide wherein said bloating is abdominal bloating.

In some embodiments, the administering of linaclotide decreases abdominal discomfort in said patient compared to said patient prior to treatment with linaclotide.

In some embodiments, the administering of linaclotide decreases abdominal pain in said patient compared to said patient prior to treatment with linaclotide.

In some aspects, the patient has very severe, severe, moderate or mild abdominal pain prior to treatment with linaclotide, wherein the administering decreases the abdominal pain in said patient compared to said patient prior to treatment with linaclotide (e.g., very severe to severe abdominal pain; severe to moderate abdominal pain; moderate to mild; mild to none; and the like).

In some embodiments, the administering of linaclotide decreases constipation severity in said patient compared to said patient prior to treatment with linaclotide.

In some embodiments, the administering of linaclotide improves stool consistency in said patient compared to said patient prior to treatment with linaclotide.

In some embodiments, the administering of linaclotide decreases straining during defecation in said patient compared to said patient prior to treatment with linaclotide.

In some embodiments, the administering of linaclotide increases the number of abdominal pain-free days in said patient compared to said patient prior to treatment with linaclotide.

In some embodiments, the administering of linaclotide improves at least two symptoms in a patient compared to said symptoms prior to linaclotide treatment, wherein the symptoms are selected from an increase in the CSBM frequency rate, an increase in the SBM frequency rate, a decrease in bloating, a decrease in abdominal discomfort, a decrease in abdominal pain, a decrease in severity of straining during defecation, or an improvement in stool consistency.

In some embodiments, the administering of linaclotide further increases the number of CSBMs by the patient to three or greater CSBMs per week.

In some embodiments, the administering of linaclotide improves patient assessment of constipation quality of life compared to the prior treatment with linaclotide.

In further embodiments, administering of linaclotide decreases abdominal symptoms (e.g., pain, discomfort, bloating) and improves bowel symptoms (e.g. CSBMs/per week, SBMs/per week, stool consistency, and straining) in a patient with greater than moderate abdominal pain prior to treatment of linaclotide.

In some embodiments, administering of linaclotide decreases abdominal symptoms (e.g. pain, discomfort, bloating) and improves bowel symptoms (e.g. CSBMs/per week, SBMs/per week, stool consistency, and straining) in a patient with moderate to severe abdominal pain prior to treatment of linaclotide.

In further embodiments, administering of linaclotide decreases abdominal symptoms (e.g. pain, discomfort, bloating) and improves bowel symptoms (e.g. CSBMs/per week, SBMs/per week, stool consistency, and straining) in a patient with severe to very severe abdominal pain prior to treatment of linaclotide.

In another aspect, the method of constipation-predominant irritable bowel syndrome according to the invention includes the absence of a symptom rebound when discontinuing the administration of a therapeutically effective dose of linaclotide.

In other embodiments, the method of treating constipation-predominant irritable bowel syndrome according to the invention includes the absence of a symptom rebound when discontinuing the administration of a therapeutically effective dose of linaclotide, wherein said symptoms are selected from a decrease in the rate of CSBMs per week, a decrease in the rate of SBMs per week, an increase in bloating, an increase in abdominal discomfort, an increase in abdominal pain, an increase in constipation severity, a decrease in stool consistency, or an increase in straining during defecation.

In further embodiments, discontinuing the administration of linaclotide does not produce a symptom rebound of weekly CSBMs for a patient.

In other embodiments, discontinuing the administration of linaclotide does not produce a symptom rebound of weekly SBMs for a patient.

In still other embodiments, discontinuing the administration of linaclotide does not produce a symptom rebound of decreased stool consistency for a patient (e.g., as measured by BSFS).

In some embodiments, discontinuing the administration of linaclotide does not produce a symptom rebound of severity of straining during defecation for a patient.

In some embodiments, discontinuing the administration of linaclotide does not produce a symptom of abdominal pain for a patient.

In other embodiments, discontinuing the administration of linaclotide does not produce a symptom rebound of bloating for a patient.

In some embodiments, discontinuing the administration of linaclotide does not produce a symptom rebound of constipation severity for a patient.

In still further embodiments, discontinuing the administration of linaclotide does not produce a symptom rebound for global relief of constipation for a patient.

In other embodiments the invention provides a method of optimizing the treatment with linaclotide of a patient with constipation-predominant irritable bowel syndrome, comprising

a) administering a first therapeutically effective dose of linaclotide once a day;

b) determining whether the patient develops loose stools or diarrhea after treatment with linaclotide;

c) wherein if the patient develops loose stools or diarrhea after one or more days of said administering, administering a second therapeutically effective dose of linaclotide once a day, wherein said second therapeutically effective dose is lower than said first therapeutically effective dose.

In some embodiments, the method of treating constipation-predominant irritable bowel syndrome according to the invention results in a 1.5 or greater fold CSBM increase compared to baseline, i.e., prior to treatment with linaclotide. In other embodiments, the fold CSBM increase is 2.0 or greater. In other embodiments, the fold CSBM increase is 2.5 or greater. In still other embodiments, the fold CSBM increase is 3.0 or greater.

In some embodiments, the method of treating constipation-predominant irritable bowel syndrome according to the invention provides a patient a 10% decrease or greater in abdominal pain symptoms compared to baseline, i.e., prior to treatment with linaclotide. In other embodiments, the decrease in abdominal pain symptoms compared to baseline is 20% or greater. In other embodiments, the decrease in abdominal pain symptoms compared to baseline is 30% or greater. In other embodiments, the decrease in abdominal pain symptoms compared to baseline is 40% or greater. In other embodiments, the decrease in abdominal pain symptoms compared to baseline is 50% or greater. In other embodiments, the decrease in abdominal pain symptoms compared to baseline is 60% or greater.

### EXAMPLES

### Example 1

### Preparation of Linaclotide

Linaclotide as described herein was prepared by solid-phase chemical synthesis and natural folding (air oxidation) by Polypeptide Laboratories (Torrance, CA). The oral linaclotide formulation was prepared by Forest Laboratories, Inc. (New York, NY).

### Example 2

### Linaclotide Formulations

The formulations used in the invention contain linaclotide or a pharmaceutically acceptable salt of linaclotide. The formulations are stable and have a sufficient shelf life for manufacturing, storing and distributing the drug. For example, the formulations have an expected shelf life of at least 12 months at room temperature storage conditions (e.g., 25° C./60 percent relative humidity (RH)) and up to at least 18 months or 24 months at room temperature storage conditions (e.g., 25° C./60 percent RH). In the formulations, greater than or equal to 95 percent of the original amount of linaclotide in the composition remains after three months when packaged samples are stored at accelerated conditions (40° C./75 percent RH) when assessed in an assay on a weight/weight basis as determined by high pressure liquid chromatography (HPLC) against a linaclotide reference standard.

The GC-C receptor agonist polypeptide formulations are prepared from a solution, e.g., an aqueous solution (“the coating solution”), comprising: (i) a GC-C receptor agonist polypeptide such as linaclotide or a pharmaceutically acceptable salt thereof; (ii) a cation selected from Mg²⁺, Ca²⁺, Zn²⁺, Mn²⁺, K⁺, Na⁺ and Al³⁺ and/or a sterically hindered primary amine (e.g., leucine); and optionally (iii) a pharmaceutically acceptable binder. The GC-C receptor agonist polypeptide formulations can optionally include one or more of a pharmaceutically acceptable glidant, a pharmaceutically acceptable lubricant or a pharmaceutically acceptable additive that acts as both a glidant and lubricant.

It has been found that a cation selected from Mg²⁺, Ca²⁺, Zn²⁺, Mn²⁺, Na and Al³⁺ is useful for suppressing the formation of an oxidation product of the GC-C receptor agonist polypeptide linaclotide during storage. It has also been found that a sterically hindered primary amine is useful for suppressing the formation of a formaldehyde imine adduct of the GC-C receptor agonist polypeptide linaclotide (“formaldehyde imine product”) during storage. Thus, the GC-C receptor agonist polypeptide formulations comprising a cation selected from Mg²⁺, Ca²⁺, Zn²⁺, Mn²⁺, K⁺, Na⁺ or Al³⁺—e.g., a divalent cation selected from Zn²⁺, Me⁺ and Ca²⁺—and/or a sterically hindered primary amine, such as an amino acid, have a sufficient shelf life (as measured by chromatographic purity and/or by a weight/weight assay) for manufacturing, storing and distributing the drug. Further, while the presence of a sterically hindered amine alone can increase the formation of a hydrolysis product of linaclotide during storage, the combination of a sterically hindered primary amine and a cation, e.g., the combination of leucine and Ca²⁺, suppresses the formation of the hydrolysis product of the GC-C receptor agonist polypeptide as well as the oxidation product of GC-C receptor agonist polypeptide during storage, leading to an even greater overall stability as determined by a weight/weight assay and/or by chromatographic purity.

GC-C receptor agonist polypeptide formulations are typically produced as follows.

### Preparation of the Coating Solution:

Approximately 8.3 kg of purified water is mixed with hydrochloric acid to create a solution with a pH between 1.5 and 2.0. The cation, if used, is added to the solution in a quantity to provide the desired concentration, and the solution is mixed for sufficient time to produce a clear solution. The sterically hindered primary amine, if used, is added to the solution in a quantity to provide the desired concentration, and the solution is mixed for sufficient time to produce a clear solution. Other additives, such as antioxidants, are then added, if desired. The binder is then added to the solution and the solution is mixed for sufficient time to achieve a clear solution. The pH of the solution is tested, and hydrochloric acid is added if necessary to produce a solution having a pH between 1.5 and 2.0. This is Solution 1.

Approximately 8.3 kg of purified water is mixed with hydrochloric acid to create a solution with a pH between 1.5 and 2.0. The desired amount of linaclotide is added to the solution and mixed for 10 to 30 minutes. The pH of the solution is tested, and hydrochloric acid is added if necessary to produce a solution having a pH between 1.5 and 2.0. This is Solution 2.

Solution 1 and Solution 2 are then mixed together. The pH of the solution is tested, and hydrochloric acid is added if necessary to produce a solution having a pH between 1.5 and 2.0. This is the coating solution.

### Preparation of the Active Beads:

Approximately 24.19 kg of microcrystalline cellulose beads are added to a Wurster Column of a Glatt GPCG-30 Fluid Bed. The microcrystalline cellulose beads are fluidized and heated to product temperature of 45-47° C. Next, the coating solution is layered to the beads. The product spraying temperature is controlled between 37° C. and 47° C. by controlling inlet temperature, spray rate, atomization pressure, and air volume. After the entire coating solution is layered to the beads, the beads are dried with a product drying temperature of 37° C. to 47° C. The product of this process is referred to as active beads.

### Example 3

### Measurement of Linaclotide Content and Purity

Linaclotide content and purity, as well as measurement of linaclotide-related substances may be determined, for example, by reverse phase gradient liquid chromatography using an Agilent Series 1100 LC System with Chemstation Rev A.09.03 software or the equivalent. A YMC Pro™ C18 column (dimensions: 3.0×150 mm, 3.5 μm, 120 Å; Waters Corp., Milford, Mass.) or the equivalent is used and is maintained at 40° C. Mobile phase A (MPA) consists of water with 0.1% trifluoroacetic acid, while mobile phase B (MPB) consists of 95% acetonitrile:5% water with 0.1% trifluoroacetic acid. Elution of linaclotide and its related substances is accomplished with a gradient from 0% to 47% MPB in 28 minutes followed by a ramp to 100% MPB in 4 minutes with a 5 minute hold at 100% MPB to wash the column. Re-equilibration of the column is performed by returning to 0% MPB in 1 minute followed by a 10 minute hold at 100% MPA. The flow rate is 0.6 mL/min and detection is accomplished by UV at 220 nm.

Samples for analysis are prepared by addition of the contents of linaclotide capsules to 0.1 N HCl to obtain a target concentration of 20 μg linaclotide/mL. 100 μL of this solution is injected onto the column.

Linaclotide content is measured by determining the linaclotide concentration in the prepared sample against a similarly prepared external linaclotide standard.

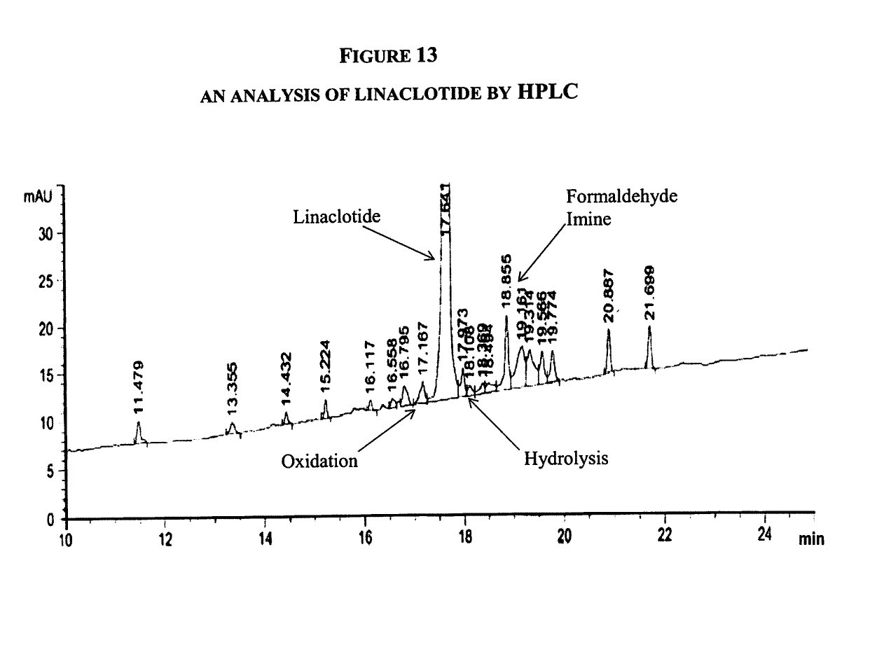

An example of an analysis of linaclotide by HPLC is shown in FIG. 13, wherein “Oxidation” refers to the linaclotide oxidation product, “Formaldehyde Imine” refers to the linaclotide formaldehyde imine product and “Hydrolysis” refers to the linaclotide hydrolysis product.

### Example 4

### Linaclotide Capsule Formulation

Example linaclotide capsule formulations were produced essentially as described in Table 1, which provides the amounts of cation, sterically hindered primary amine, binder, linaclotide and beads, and their theoretical weights (mg/g) and (kg/Batch) for the complete linaclotide beads drug layer solution. Table 2 provides the conditions under which the beads were coated. Table 3 provides the ingredients and theoretical weights (mg/g) and (kg/Batch) for the preparation of the linaclotide active beads.

The linaclotide active beads were tested for linaclotide content. Based on the assay of the active beads, an appropriate amount of active beads was filled into size 2 hard gelatin capsules (weight 61 mg), using an MG2 Futura encapsulation machine, to achieve the desired linaclotide concentration. The 300 μg linaclotide capsules contained 113 mg linaclotide beads (600 μg linaclotide/225 mg beads) having an effective linaclotide content of 266 μg. The linaclotide content can be measured, for example, by using the assay described in Example 3 or by other methods.

TABLE 1
Example linaclotide capsule formulation

| | | Theoretical | Theoretical |
|---|---|---|---|
| | | Weight | Weight |
| Ingredients | Function | (mg/g) | (kg/batch) |
| Linaclotide | API | 2.67 | 0.067 |
| CaCl₂•2H₂O, USP, | Stabilizer | 15.41 | 0.385 |
| EP, BP, JP | | | |
| L-Leucine, USP | Stabilizer | 6.87 | 0.172 |
| Hydroxypropyl | Binder | 7.00 | 0.175 |
| Methylcellulose, | | | |
| USP (Methocel | | | |
| E5 Premium LV) | | | |
| Purified Water, USP | — | — | 16.666 |
| HCl (36.5-38.0), NF | — | — | 0.114 |

TABLE 2
Example Bead Coating Conditions

| | | | Atomi- | Process | Product |
|---|---|---|---|---|---|
| Product | Inlet | Spray | zation | Air | Drying |
| Spraying | Temp | rate | Pressure | Volume | Temp |
| Temp (° C.) | (° C.) | (g/min) | (bar) | (cfm) | (° C.) |
| 64.9-65.1 | 80 | 150 | 2.0 | 515-564 | 54.9-55.0 |

TABLE 3
Example ingredients and theoretical
weights for linaclotide active beads

| | | Theoretical | Theoretical |
|---|---|---|---|
| | | Weight | Weight |
| Ingredients | Function | (mg/g) | (kg/batch) |
| Linaclotide Beads | Coating | 31.95 | 0.799 |
| Drug Layer | solution | | |
| Solution | | | |
| Microcrystalline | Beads | 968.05 | 24.201 |
| cellulose | | | |
| spheres NF | | | |
| (Celphere CP-305) | | | |
| Final Total: | Active | 1000 | 25.000 |
| Linaclotide Beads, | beads | | |
| 600 μg/225 mg) | | | |

### Example 5

### Administration of Linaclotide for the treatment of Constipation-Predominant Irritable Bowel Syndrome

Linaclotide capsules from Example 4 were administered in two multicenter, randomized, double-blind, placebo-controlled parallel-group trials.

### Constipation-Predominant Irritable Bowel Syndrome Trial 1 (LIN-MD-31):

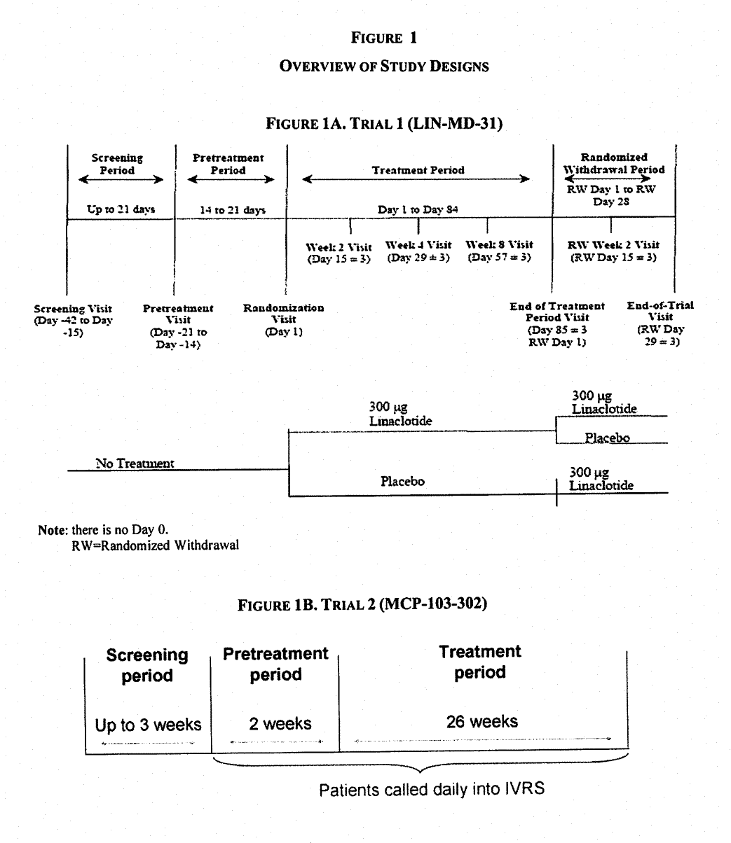

Trial 1 (LIN-MD-31) was conducted on approximately 800 patients meeting modified Rome II criteria for constipation-predominant irritable bowel syndrome. The trial included a two-week pretreatment baseline period, a 12-week treatment period, and a randomized withdrawal period (see FIG. 1A).

### Constipation-Predominant Irritable Bowel Syndrome Trial 2 (MCP-103-302):

Trial 2 (MCP-103-302) was conducted on approximately 805 patients meeting modified Rome II criteria for constipation-predominant irritable bowel syndrome (<3 CSBM/wk, ≤5 SBM/wk, abdominal pain ≥3 on a 0-10 scale.) As in Trial 1, the trial included a two-week pretreatment baseline period; however, the treatment period was continued for 26 weeks rather than 12 weeks as in Trial 1, and no randomized withdrawal period was performed (see FIG. 1B).

### Pretreatment (Baseline) Period:

The Pretreatment Period is defined as the 14 to 21 calendar days immediately before starting the trial during which patients provided information related to their daily bowel habits, daily assessment of the symptom severity (such as, e.g., abdominal pain), constipation severity, and use of other medicines, laxatives, suppositories, and/or enemas. Patients who satisfied the necessary criteria were entered into the Treatment Period.

During the two-week pretreatment period, approximately 76 percent of patients in Trial 1 had no CSBMs. A CSBM is a bowel movement that occurs in the absence of laxative, enema, or suppository usage within the preceding 24 hours that is accompanied by the patient self-reporting a feeling of complete emptying of the bowel. The mean abdominal pain score was 5.6 (on a 11-point scale scored 0-10, where 0 is no abdominal pain, and 10 very severe abdominal pain). During the pre-treatment period, 88 percent of patients suffered from abdominal pain every day.

In Trial 2, the mean abdominal pain score was 5.6 (on a 11-point scale scored 0-10, where 0 is no abdominal pain, and 10 very severe abdominal pain). During the pre-treatment period, 87 percent of patients suffered from abdominal pain every day. Approximately 76% of patients had no CSBMs.

### Treatment Period:

In both trials, the Treatment Period began with randomization. In Trial 1, treatment lasted for 12 weeks. In Trial 2, treatment lasted for 26 weeks. Patients were randomized to approximately equal-sized treatment groups consisting of 266 μg linaclotide or a placebo, taken once daily in the morning 30 minutes before breakfast. Patients continued to provide daily assessments such as their daily bowel habit assessments and daily patient symptom severity assessments.

In both trials, daily assessment data for patients in both treatment groups were compiled to give the following primary, co-primary and secondary treatment efficacy endpoints, which have been defined by United States and European regulatory agencies for the study of the efficacy of IBS-C.

### Primary Efficacy Endpoints:

A patient was classified as a 9/12 Week APC 3+1 Responder if they were a weekly APC 3+1 Responder for ≥9 out of the 12 (i.e., 9/12) weeks of the study.

A patient was classified as a 9/12 Week CSBM 3+1 Responder if they were a weekly CSBM 3+1 Responder for ≥9/12 weeks.

A patient was classified as a 9/12 Week Abdominal Pain Responder if they were a weekly Abdominal Pain Responder for ≥9/12 weeks.

A patient was classified as a 6/12 Week APC+1 Responder if they were a weekly APC +1 Responder for ≥6 out of the 12 (i.e., 6/12) weeks of the study.

### Co-Primary Efficacy Parameters

A patient was classified as a 12-week Abdominal Pain/Abdominal Discomfort Responder if they had a decrease of ≥30% in either the mean abdominal pain score or mean abdominal discomfort score with neither score worsening from baseline for 6/12 weeks.

A patient was classified as a 12-week IBS Degree of Relief Responder if they had an assessment of “considerably relieved” or “completely relieved” for ≥6/12 weeks.

### Secondary Efficacy Endpoints:

In Trial 2, the above co-primary efficacy parameters, 12-week Abdominal Pain/Abdominal Discomfort Responder and 12-week IBS Degree of Relief Responder, were also computed for the full 26 week treatment period.

Also compiled from the patient daily data were the following secondary efficacy endpoints:

Change From Baseline (i.e., change from pre-treatment period) in CSBM Frequency Rate (measured as a weekly rate);

Change From Baseline (“CFB”) in SBM Frequency Rate (weekly rate);

Change From Baseline in Stool Consistency (measured on a 7-point BSFS);

Change From Baseline in Severity of Straining (measured on a 5-point scale);

Change From Baseline in Abdominal Pain (measured on a 11-point numerical rating scale and scored 0-10);

Change From Baseline in Abdominal Discomfort (measured on all-point numerical rating scale and scored 0-10);

Change From Baseline in Bloating (measured on a 11-point numerical rating scale and scored 0-10);

6/12 Week CSBM+1 Responder;

6/12 Week Abdominal Pain Responder; and

Change From Baseline in Percent of Abdominal Pain-free Days (Pain-free is a score of 0 on the 0-10 point scale).

### Results of Trial 1:

TABLE 4
Primary and secondary efficacy endpoints

| | Statistical | |
|---|---|---|
| | Significance* | |
| | (266 μg vs. | Nominal |
| Parameter | Placebo) | P-value |
| Primary efficacy endpoints | | |
| 9/12 Week APC 3 + 1 Responder | Yes | 0.0004 |
| 9/12 Week CSBM 3 + 1 Responder | Yes | <0.0001 |
| 9/12 Week Abdominal Pain Responder | Yes | 0.0262 |
| 6/12 Week APC + 1 Responder | Yes | <0.0001 |
| Secondary efficacy endpoints | | |
| 12-Week CSBM Frequency Rate (CFB) | Yes | <0.0001 |
| 12-Week SBM Frequency Rate (CFB) | Yes | <0.0001 |
| 12-Week Stool Consistency (CFB) | Yes | <0.0001 |
| 12-Week Severity of Straining (CFB) | Yes | <0.0001 |
| 12-Week Abdominal Pain (CFB) | Yes | <0.0001 |
| 12-Week Abdominal Discomfort (CFB) | Yes | <0.0001 |
| 12-Week Bloating (CFB) | Yes | <0.0001 |
| 6/12 Week CSBM + 1 Responder | Yes | <0.0001 |
| 6/12 Week Abdominal Pain Responder | Yes | 0.0003 |
| 12-Week Percent of Abdominal | Yes | 0.0014 |
| Pain-free Days (CFB) | | |

TABLE 5
Co-Primary Efficacy Parameters

| | Statistical | |
|---|---|---|
| | Significance | |
| | (266 μg vs. | Nominal |
| Parameter | Placebo) | P-value |
| 12-week Abdominal | Yes | =0.0002 |
| Pain/Abdominal | | |
| Discomfort Responder | | |
| 12-week IBS Degree | Yes | <0.0001 |
| of Relief Responder | | |

Analysis of the data, shown in Table 4, clearly indicate clinically meaningful and statistically significant improvement was achieved for linaclotide-treated patients compared to placebo-treated patients for all four primary efficacy endpoints.

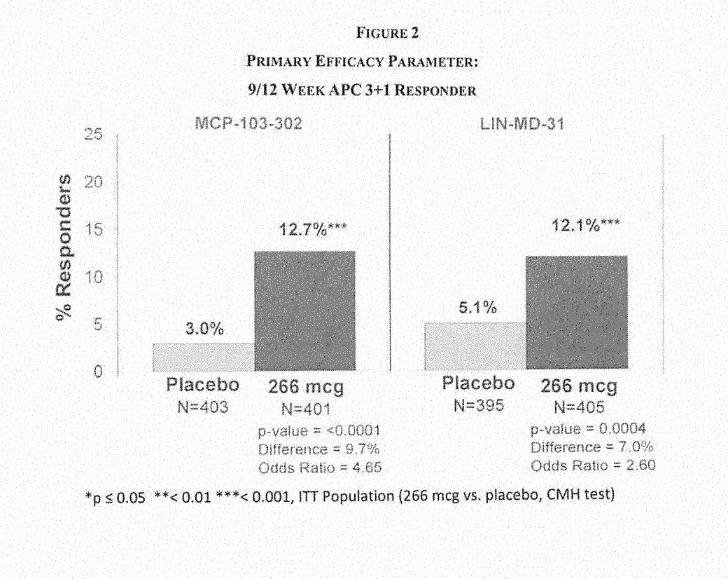

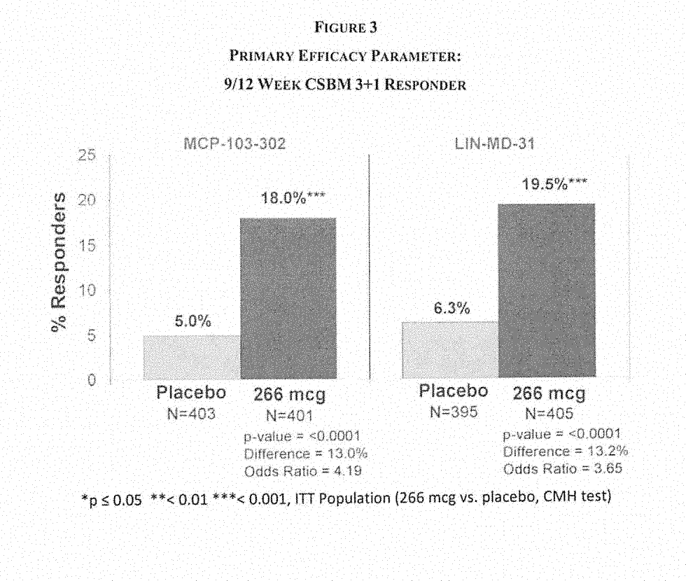

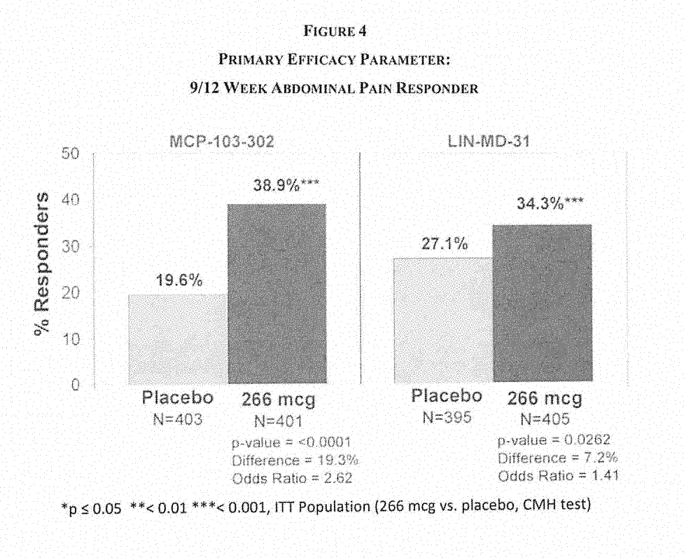

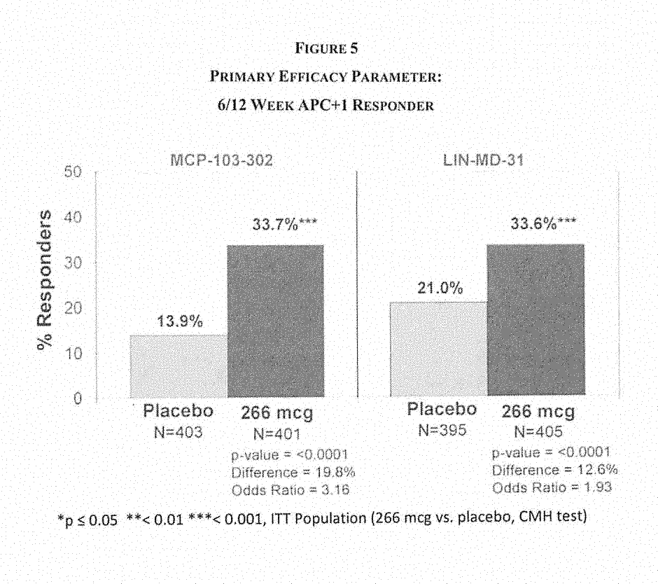

Linaclotide-treated patients demonstrated a significant increase of the 9/12-week APC 3+1 responder rate (p=0.0004) which was 12.1 percent in the 266 μg linaclotide group, as compared to 5.1 percent in the placebo group (FIG. 2). Linaclotide-treated patients also demonstrated a significant increase in 9/12-week CSBM 3+1 responder rate (p<0.0001) which was 19.5 percent in the 266 μg linaclotide group, as compared to 6.3 percent in the placebo group (FIG. 3). Linaclotide-treated patients demonstrated a significant increase 9/12-week abdominal pain responder rate (p=0.0262) which was 34.3 percent in the 266 μg linaclotide group, as compared to 27.1 percent in the placebo group (FIG. 4). Finally, linaclotide-treated patients demonstrated a significant increase 6/12-week APC+1 pain responder rate (p<0.0001) which was 33.6 percent in the 266 μg linaclotide group, as compared to 21.0 percent in the placebo group (FIG. 5).

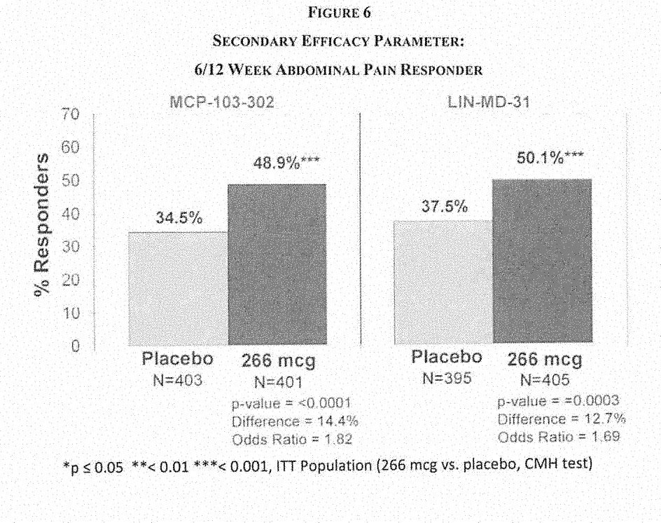

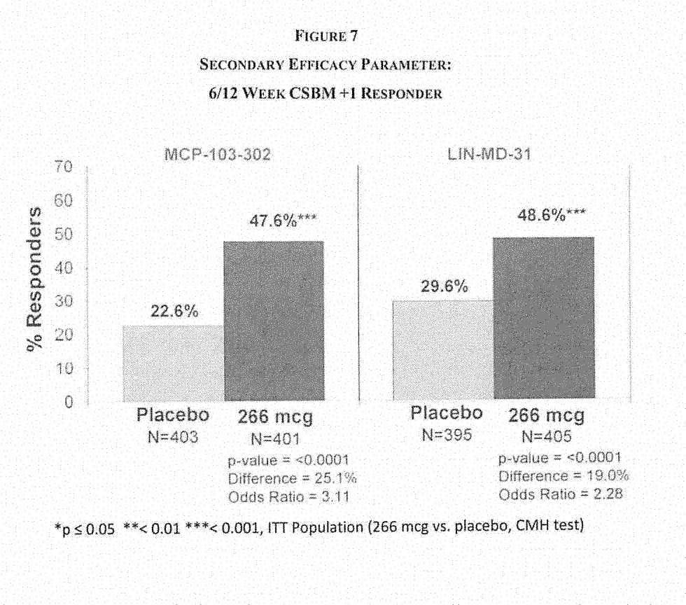

All secondary endpoints measured in the study also showed significant improvements (p<0.0014) for linaclotide-treated patients, as seen in Table 4. Linaclotide-treated patients demonstrated a significant increase in 6/12-week abdominal pain responder rate (p=0.0003), which was 50.1 percent in the 266 μg linaclotide group, as compared to 37.5 percent in the placebo group (FIG. 6); and a significant increase in 6/12 week CSBM+1 responder rate (p<0.0001), which was 48.6 percent in the 266 μg linaclotide group, as compared to 29.6 percent in the placebo group (FIG. 7).

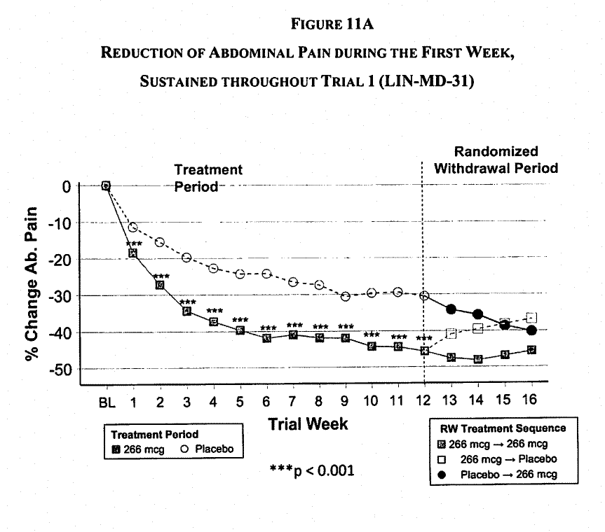

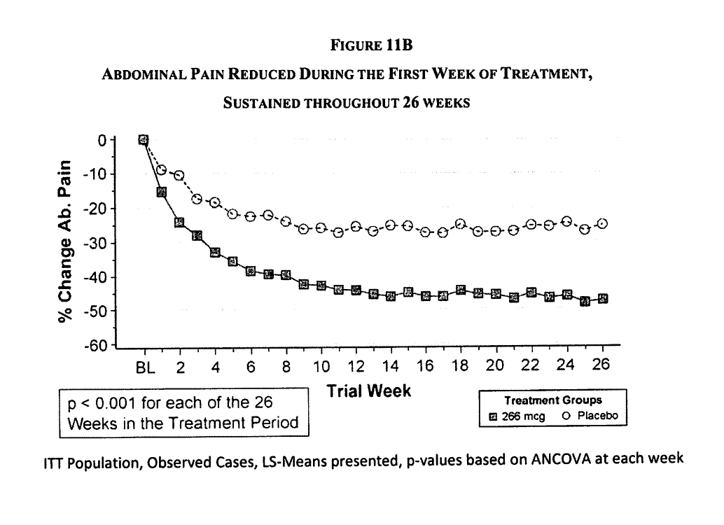

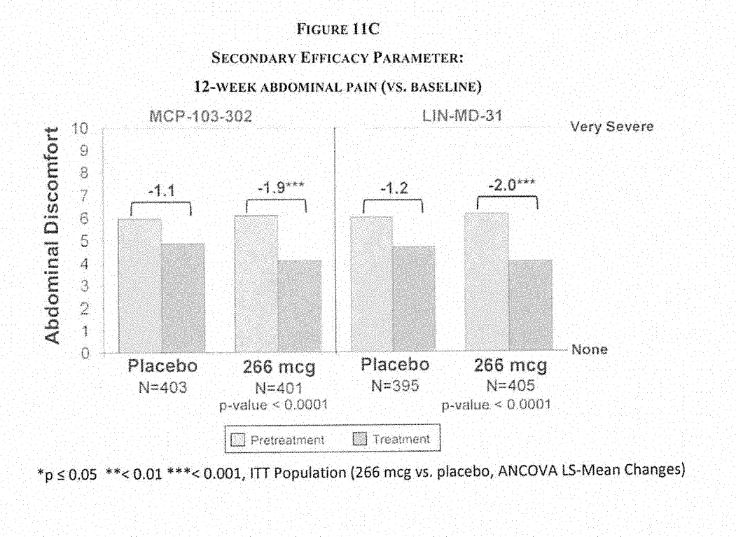

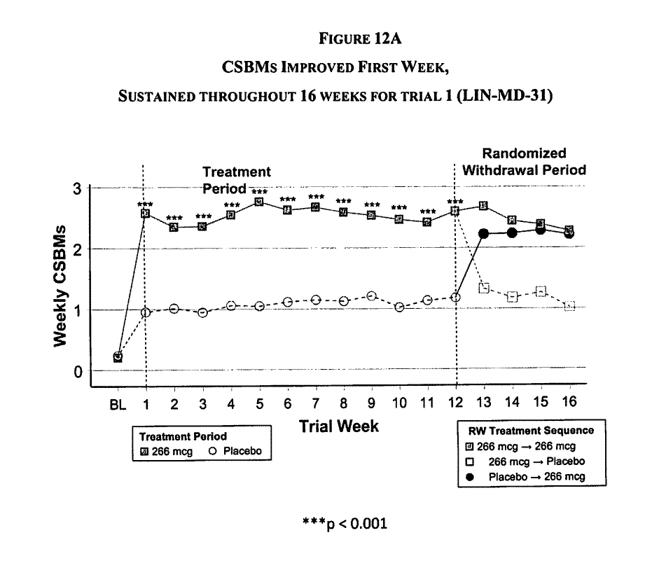

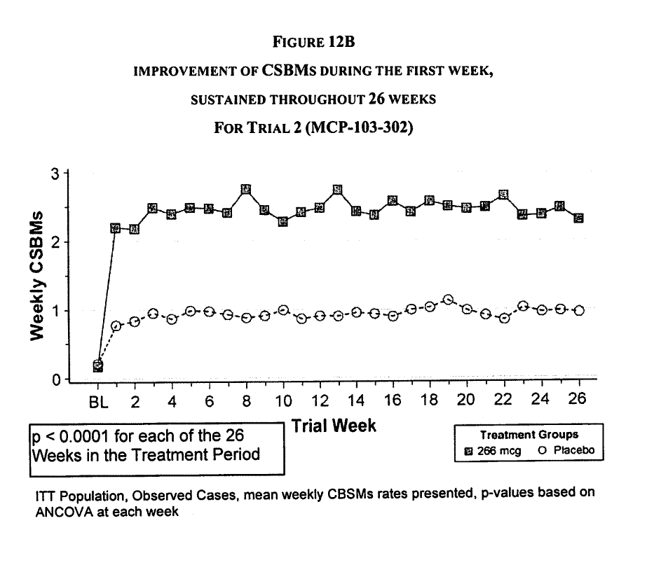

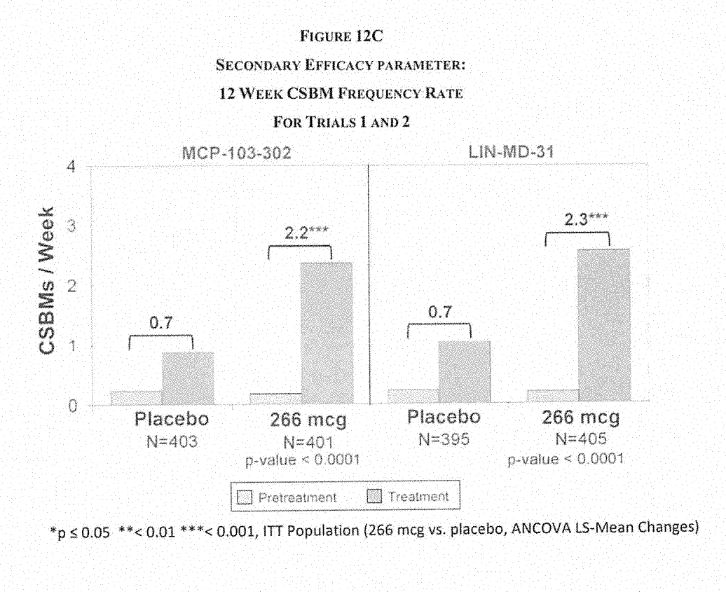

Also, as shown in Table 4, the patients in the trial also exhibited statistically significant changes (all at p<0.0001) in CSBM frequency rate (FIG. 12C, discussed below), SBM frequency rate, stool consistency, severity of straining, abdominal pain (FIG. 11C, discussed below), abdominal discomfort, bloating, and 6/12 week CSBM+1 responder for linaclotide versus placebo.

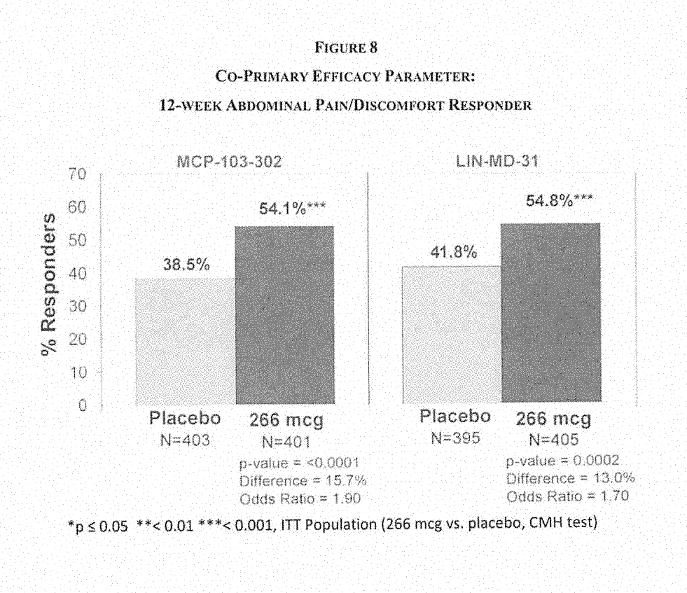

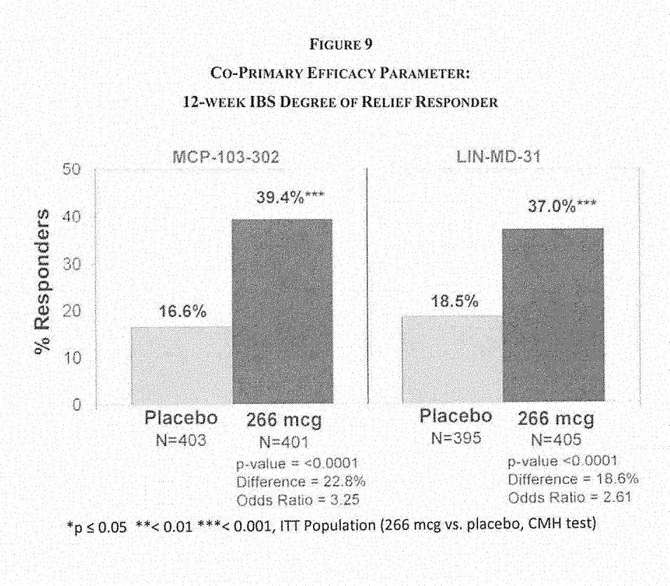

As seen in Table 5, there were significant differences in Co-Primary Efficacy Parameters as well between the linaclotide and placebo treatments. Linaclotide-treated patients demonstrated a significant increase in the 12-week Abdominal Pain/Abdominal Discomfort Responder (p=0.0002), which was 54.8 percent in the 266 μg linaclotide group, as compared to 41.8 percent in the placebo group (FIG. 8). Similarly, linaclotide-treated patients demonstrated a significant increase over the placebo in the 12-week IBS Degree of Relief Responder (p<0.0001), which was 37.0 percent in the 266 μg linaclotide group, as compared to 18.5 percent in the placebo group (FIG. 9).

### Randomized Withdrawal Period:

Patients who were randomized to linaclotide in the Treatment Period and completed the 12 weeks of the Treatment Period, the RW Population, were randomized to treatment with linaclotide or placebo in the RW Period as follows: half of the RW Population received placebo for the first half of the RW Period followed by administration of 266 μg linaclotide; one quarter of the RW Population received 266 μg linaclotide for the entire RW Period and the remaining one quarter of the RW Population received 266 μg linaclotide for the first half of the RW Period and placebo for the remainder of the RW Period. Patients that were randomized to placebo in the Treatment Period and completed the 12 weeks of the Treatment Period received 266 μg linaclotide in the RW Period.

The results from Randomized Withdrawal Period additionally demonstrate that patients initiating linaclotide treatment had marked improvement in IBS-C symptoms. There was no evidence of rebound of IBS-C symptoms or increase in the frequencies of adverse events such as abdominal pain or bowel movement symptoms following discontinuation of linaclotide treatment. The incidence of adverse events in patients initiating linaclotide treatment was similar to the incidence in those receiving linaclotide during the first 4 weeks of the Treatment Period. Patients continuing linaclotide treatment showed sustained improvements in bowel and abdominal symptoms and global assessments.

The most common adverse events that occurred more frequently in linaclotide-treated patients compared to placebo-treated patients were diarrhea (19 percent vs. 4 percent: with 9% linaclotide group/2% placebo group with mild symptoms; 8%/2% with moderate symptoms; and 2%/0.3% with severe symptoms), flatulence (5 percent vs. 2 percent), abdominal pain (5 percent vs. 3 percent), and headache (5 percent vs. 4 percent). Overall rates of discontinuation due to adverse events were 8 percent for the linaclotide group and 3 percent for the placebo group. The most common reason for discontinuation in linaclotide-treated patients was due to gastrointestinal related adverse events.

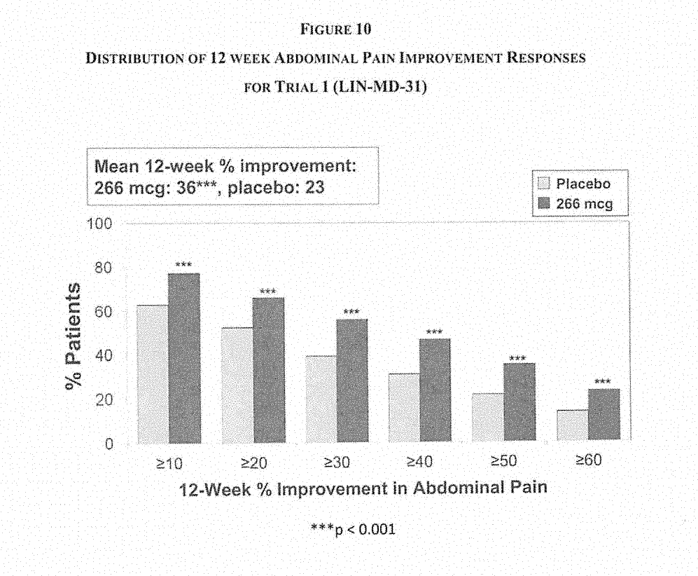

As seen in FIG. 10, just under 80% of patients reported at least a 10% improvement in abdominal symptoms over 12 weeks with the linaclotide treatment over the placebo (about 60%), which was significantly larger (p<0.001). Significantly more patients reported 20%, 30%, 40%, 50%, and 60% abdominal pain improvements over the course of the study with the linaclotide treatment over the placebo (see FIG. 10).

For patients taking linaclotide, on average, abdominal pain was reduced over the pre-treatment baseline within 1-3 days of treatment and sustained throughout the treatment period (FIG. 11A); the decrease was significantly larger (p<0.001) than with the placebo alone (see FIG. 11C).

On average, patients taking linaclotide had about 1.5 more CSBMs per week than with the placebo alone, which was statistically significant (p<0.001; FIG. 12C); the CSBM improved over baseline the first week after the beginning of treatment and continued throughout the study (see FIG. 12A). The results were consistent with previous clinical studies. The tolerability of the linaclotide was consistent with previous trials, as well.

### Results of Trial 2:

TABLE 6
Primary and secondary efficacy endpoints

| | Statistical | |
|---|---|---|
| | Significance | |
| | (266 μg vs. | Nominal |
| Parameter | Placebo) | P-value |
| Primary efficacy endpoints | | |
| 9/12 Week APC 3 + 1 Responder | Yes | <0.0001 |
| 9/12 Week CSBM 3 + 1 Responder | Yes | <0.0001 |
| 9/12 Week Abdominal | Yes | <0.0001 |
| Pain Responder | | |
| 6/12 Week APC + 1 Responder | Yes | <0.0001 |
| Secondary efficacy endpoints | | |
| 12-Week CSBM Frequency Rate (CFB) | Yes | <0.0001 |
| 12-Week SBM Frequency Rate (CFB) | Yes | <0.0001 |
| 12-Week Stool Consistency (CFB) | Yes | <0.0001 |
| 12-Week Severity of Straining (CFB) | Yes | <0.0001 |
| 12-Week Abdominal Pain (CFB) | Yes | <0.0001 |
| 12-Week Abdominal Discomfort (CFB) | Yes | <0.0001 |
| 12-Week Bloating (CFB) | Yes | <0.0001 |
| 6/12 Week CSBM + 1 Responder | Yes | <0.0001 |
| 6/12 Week Abdominal Pain Responder | Yes | <0.0001 |
| 12-Week Percent of Abdominal | Yes | =0.0003 |
| Pain-free Days (CFB) | | |

TABLE 7
Co-Primary Efficacy Parameters

| | Statistical | |
|---|---|---|
| | Significance | |
| | (266 μg vs. | Nominal |
| Parameter | Placebo) | P-value |
| 12-week Abdominal | Yes | <0.0001 |
| Pain/Abdominal | | |
| Discomfort Responder | | |
| 12-week IBS Degree | Yes | <0.0001 |
| of Relief Responder | | |
| Main Secondary | | |
| efficacy endpoints | | |
| 26-week Abdominal | Yes | <0.0001 |
| Pain/Abdominal | | |
| Discomfort Responder | | |
| 26-week IBS Degree | Yes | <0.0001 |
| of Relief Responder | | |

Analysis of the data, shown in Table 6, clearly indicate clinically meaningful and statistically significant improvement was achieved for linaclotide-treated patients compared to placebo-treated patients for all four primary efficacy endpoints.

Linaclotide-treated patients demonstrated a significant increase of the 9/12-week APC 3+1 responder rate (p<0.0001), which was 12.7 percent in the 266 μg linaclotide group as compared to 3.0 percent in the placebo group (FIG. 2). Linaclotide-treated patients also demonstrated a significant increase in 9/12-week CSBM 3+1 responder rate (p<0.0001), which was 18.0 percent in the 266 μg linaclotide group as compared to 5.0 percent in the placebo group (FIG. 3). Linaclotide-treated patients demonstrated a significant increase 9/12-week abdominal pain responder rate (p<0.0001), which was 38.9 percent in the 266 μg linaclotide group as compared to 19.6 percent in the placebo group (FIG. 4). Finally, linaclotide-treated patients demonstrated a significant increase 6/12-week APC+1 pain responder rate (p<0.0001), which was 33.7 percent in the 266 μg linaclotide group as compared to 13.9 percent in the placebo group (FIG. 5).

As seen in Table 6, all secondary endpoints measured in the study also showed significant improvements (p<0.0001) for linaclotide-treated patients which included the 6/12 Abdominal pain Responder (FIG. 6), the 6/12 week CSBM+1 Responder (FIG. 7), abdominal pain (FIG. 11C), the 12-Week CSBM Frequency Rate (CFB; FIG. 12C), 12-Week Stool Consistency (CFB), 12-Week Severity of Straining (CFB), and 12-Week Bloating (CFB). The 12 week percent of abdominal pain-free (CFB) days was also statistically significant (p=0.0003).

All co-primary endpoints measured in the study also showed significant improvements (p<0.0001) for linaclotide-treated patients, as seen in Table 7.

As seen in Table 7, there were significant differences in co-primary efficacy Parameters as well between the linaclotide and placebo treatments. Linaclotide-treated patients demonstrated a significant increase in the 12-week Abdominal Pain/Abdominal Discomfort Responder (p<0.0001), which was 54.1 percent in the 266 μg linaclotide group, as compared to 38.5 percent in the placebo group (FIG. 8). Similarly, linaclotide-treated patients demonstrated a significant increase over the placebo in the 12-week IBS Degree of Relief Responder (p<0.0001), which was 39.4 percent in the 266 μg linaclotide group, as compared to 16.6 percent in the placebo group (FIG. 9). As also seen in Table 7, all main secondary endpoints measured in the study also showed significant improvements (p<0.0001) for linaclotide-treated patients which included the 26-week Abdominal Pain/Abdominal Discomfort Responder and the 26-week IBS Degree of Relief Responder.

The most common adverse events that occurred more frequently in linaclotide-treated patients compared to placebo-treated patients after 12 weeks were diarrhea (18 percent vs. 2 percent, flatulence (3 percent vs. 2 percent), abdominal pain (4 percent vs. 3 percent), and headache (3 percent vs. 2 percent). Overall rates of discontinuation after 12 weeks due to adverse events were 9 percent for the linaclotide group and 2 percent for the placebo group. The most common reason for discontinuation in linaclotide-treated patients was due to gastrointestinal related adverse events.

The most common adverse events that occurred more frequently in linaclotide-treated patients compared to placebo-treated patients throughout the 26-week treatment period were diarrhea (19.7 percent vs. 2.5 percent), abdominal pain (4.5 percent vs. 4.0 percent), flatulence (3.7 percent vs. 2.2 percent), viral gastroenteritis (3.7 percent vs. 2.2 percent), and headache (3.2 percent vs.2.7 percent). Overall rates of discontinuation due to adverse events were 10.2 percent for the linaclotide-treated patients and 2.5 percent for the placebo-treated patients; 4.5 percent of linaclotide-treated patients discontinued due to diarrhea compared with 0.2 percent of placebo-treated patients.

For patients taking linaclotide, on average, abdominal pain was reduced over the pre-treatment baseline within 1-3 days of treatment and sustained throughout the 26 week treatment period (FIG. 11B). After 12 weeks, the decrease was significantly larger (p<0.0001) than with the placebo alone. (FIG. 11C).

On average, patients taking linaclotide had more CSBMs per week than with the placebo alone, which was statistically significant (p<0.0001) 12 weeks after treatment (FIG. 12C). On average, CSBMs were increased over the pre-treatment baseline after a week of treatment, which was sustained throughout the 26 week treatment period (FIG. 12B).

The results were consistent with previous clinical studies including Trial 1. The tolerability of the linaclotide was consistent with previous trials, as well.

### Other Embodiments

All publications and patents referred to in this disclosure are incorporated herein by reference to the same extent as if each individual publication or patent application were specifically and individually indicated to be incorporated by reference. Should the meaning of the terms in any of the patents or publications incorporated by reference conflict with the meaning of the terms used in this disclosure, the meaning of the terms in this disclosure are intended to be controlling. Furthermore, the foregoing discussion discloses and describes merely exemplary embodiments of the present invention. One skilled in the art will readily recognize from such discussion and from the accompanying drawings and claims, that various changes, modifications and variations can be made therein without departing from the spirit and scope of the invention as defined in the following claims.

What is claimed is:

1. A method of treating a patient with constipation-predominant irritable bowel syndrome, comprising administering a therapeutically effective dose of linaclotide once a day.

2. The method according to claim 1, wherein the therapeutically effective dose is administered once a day in the morning.

3. The method according to claim 2, wherein the therapeutically effective dose is administered at least 30 minutes before ingestion of food.

4. The method according to claim 2, wherein the therapeutically effective dose is administered at least 30 minutes before breakfast.

5. The method according to claim 2, wherein the therapeutically effective dose is administered at least 120 minutes before ingestion of food.

6. The method according to any one of claim 1-5, wherein the therapeutically effective dose is 100 to 600 μg linaclotide.

7. The method according to claim 6, wherein the therapeutically effective dose is 200 μg to 300 μg linaclotide.

8. The method according to claim 6, wherein the therapeutically effective dose is 266 μg linaclotide.

9. The method according to any one of claims 1-8, wherein the linaclotide is administered for a period of greater than four weeks.

10. The method according to claim 9, wherein the linaclotide is administered for a period of at least 12 weeks.

11. The method according to any of claims 1-10, wherein the linaclotide is administered each day of the week.

12. The method according to any of claims 1-10, wherein the linaclotide is administered at least once a week, at least twice a week, at least three times a week, at least four times a week, at least five times a week or at least six times a week.

13. The method according to any one of claims 1-12, wherein the linaclotide is provided in a formulation comprising
(a) linaclotide or pharmaceutically acceptable salts thereof;
(b) CaCl₂;
(c) L-Leucine; and
(d) Hydroxypropyl Methylcellulose
wherein linaclotide is present in the formulation in an amount between 100 μg to 600 μg and the molar ratio of Ca²⁺:leucine:linaclotide is between 5-100:5-50:1.

14. The method of claim 13, wherein the formulation contains 133 μg of linaclotide.

15. The method of claim 13, wherein the formulation contains 266 μg of linaclotide.

16. The method according to any one of claims 13-15, wherein the linaclotide is provided as a capsule or tablet.

17. The method according to claim 16, wherein the linaclotide is provided as a capsule.

18. The method according to any one of claims 1-17, wherein the administering decreases abdominal pain in said patient compared to said patient prior to treatment with linaclotide.

19. The method according to any one of claims 1-18, wherein the administering increases the number of complete spontaneous bowel movements (CSBMs) by the patient to three or greater CSBMs per week.

20. The method according to any one of claims 1-19, wherein the administering increases the number of CSBMs by the patient by at least one CSBM per week compared to said patient prior to treatment with linaclotide.

21. The method according to any one of claims 1-20, wherein the administering increases the number of CSBMs by the patient to three or greater CSBMs per week and increases the number of CSBMs by the patient by at least one CSBM per week compared to the number of CSBMs by said patient prior to treatment with linaclotide.

22. The method according to any one of claims 1-21, wherein the administering:
decreases abdominal pain in said patient compared to said patient prior to treatment with linaclotide;
increases the number of CSBMs by the patient to three or greater CSBMs per week; and increases the number of CSBMs by the patient by at least one CSBM per week compared to the number of CSBMs by said patient prior to treatment with linaclotide.

23. The method according to any one of claims 1-22, wherein the administering increases the CSBM frequency rate in said patient compared to said patient prior to treatment with linaclotide.

24. The method according to any one of claims 1-23, wherein the administering increases the SBM frequency rate in said patient compared to said patient prior to treatment with linaclotide.

25. The method according to any one of claims 1-24, wherein the administering decreases the severity of straining during defecation in said patient compared to said patient prior to treatment with linaclotide.

26. The method according to any one of claims 1-25, wherein the administering decreases bloating in said patient compared to said patient prior to treatment with linaclotide.

27. The method according to claim 26, wherein said bloating is abdominal bloating.

28. The method according to any one of claims 1-27, wherein the administering decreases abdominal discomfort in said patient compared to said patient prior to treatment with linaclotide.

29. The method according to any one of claims 1-28, wherein the administering decreases constipation severity in said patient compared to said patient prior to treatment with linaclotide.

30. The method according to any one of claims 1-29, wherein the administering improves stool consistency in said patient compared to said patient prior to treatment with linaclotide.

31. The method according to any one of claims 1-30, wherein the administering decreases straining during defecation in said patient compared to said patient prior to treatment with linaclotide.

32. The method according to any one of claims 1-31, wherein the administering increases the number of abdominal pain-free days in said patient compared to said patient prior to treatment with linaclotide.

33. The method according to any one of claims 1-32, wherein the administering improves at least two symptoms in a patient compared to said symptoms prior to linaclotide treatment, wherein the symptoms are selected from an increase in the CSBM frequency rate, an increase in the SBM frequency rate, a decrease in bloating, a decrease in abdominal discomfort, a decrease in abdominal pain, a decrease in severity of straining during defecation, or an improvement in stool consistency.

34. The method according to claim 33, wherein said administering further increases the number of CSBMs by the patient to three or greater CSBMs per week.

35. The method according to any one of claims 1-34, wherein the administering improves patient assessment of constipation quality of life compared to the prior treatment with linaclotide.

36. The method according to any one of claims 1-35, wherein the administering of linaclotide provides sustained relief from symptoms of constipation predominant irritable bowel syndrome.

37. The method according to claim 36, wherein the administering of linaclotide provides sustained relief from symptoms of constipation predominant irritable bowel syndrome for at least 16 weeks.

38. The method according to claim 36, wherein the administering of linaclotide provides sustained relief from symptoms of constipation predominant irritable bowel syndrome for at least 1 out of 2 weeks.

39. The method according to claim 36, wherein the administering of linaclotide provides sustained relief of symptoms of constipation predominant irritable bowel syndrome for at least 3 out of 4 weeks.

40. The method according to claim 36, wherein the administering of linaclotide provides sustained relief of symptoms of constipation predominant irritable bowel syndrome for at least 6 out of 12 weeks.

41. The method according to claim 36, wherein the administering of linaclotide provides sustained relief of symptoms of constipation predominant irritable bowel syndrome for at least 9 out of 16 weeks.

42. The method according to any one of claims 1-41, wherein discontinuing the administration of a therapeutically effective dose of linaclotide does not produce a rebound of the symptoms in said patient.

43. The method according to any one of claims 1-42, wherein discontinuing the administration of a therapeutically effective dose of linaclotide does not produce a symptom rebound for the patient, wherein said symptom is selected from a decrease in the number of CSBMs per week, a decrease in the number of SBMs per week, an increase in bloating, an increase in abdominal discomfort, an increase in abdominal pain, an increase in constipation severity, a decrease in stool consistency, or an increase in straining during defecation.

44. The method according to any one of claims 1-43, wherein discontinuing the administration of linaclotide does not produce a symptom rebound of a decrease in CSBMs per week for said patient.

45. The method according to any one of claims 1-44, wherein discontinuing the administration of linaclotide does not produce a symptom rebound of a decrease in SBMs per week for said patient.

46. The method according to any one of claims 1-45, wherein discontinuing the administration of linaclotide does not produce a symptom rebound of decreased stool consistency for said patient.

47. The method according to any one of claims 1-46, wherein discontinuing the administration of linaclotide does not produce a symptom rebound of increased straining during defecation for said patient.

48. The method according to any one of claims 1-47, wherein discontinuing the administration of linaclotide does not produce a symptom rebound of increased abdominal discomfort for said patient.

49. The method according to any one of claims 1-48, wherein discontinuing the administration of linaclotide does not produce a symptom rebound of increased abdominal pain for said patient.

50. The method according to any one of claims 1-49, wherein discontinuing the administration of linaclotide does not produce a symptom rebound of bloating for said patient.

51. The method according to any one of claims 1-50, wherein discontinuing the administration of linaclotide does not produce a symptom rebound of constipation severity for said patient.

52. The method according to any one of claims 1-51, wherein discontinuing the administration of linaclotide does not produce a symptom rebound for global relief of constipation for said patient.

53. A method of optimizing the treatment with linaclotide of a patient with constipation-predominant irritable bowel syndrome, comprising:
a) administering a first therapeutically effective dose of linaclotide once a day;
b) determining whether the patient develops loose stools or diarrhea after treatment with linaclotide;
c) wherein if the patient develops loose stools or diarrhea after one or more days of said administering, administering a second therapeutically effective dose of linaclotide once a day, wherein said second therapeutically effective dose is lower than said first therapeutically effective dose.

54. The method according to claim 53, wherein the first therapeutically effective dose of linaclotide is 266 μg and the second therapeutically effective dose of linaclotide is 133 μg.

55. A method of treating a patient with constipation predominant irritable bowel syndrome, comprising administering a therapeutically effective dose of a GC-C agonist and wherein discontinuing the administration of a therapeutically effective dose of the GC-C agonist does not produce a symptom rebound of constipation predominant irritable bowel syndrome for said patient.

56. A method of treating a patient with constipation predominant irritable bowel syndrome, comprising administering a therapeutically effective dose of a GC-C agonist, wherein the GC-C agonist produces a rapid or sustained relief of symptoms to constipation predominant irritable bowel syndrome.

57. The method according claim 56, wherein the rapid relief occurs within one week.

58. The method according claim 57, wherein the rapid relief occurs within three days.

59. The method according claim 58, wherein the rapid relief occurs within two days.

60. The method according claim 59, wherein the rapid relief occurs within one day.

61. The method according to claim 56, wherein the sustained relief occurs for at least 16 weeks.

62. The method according to claim 56, wherein the sustained relief occurs for at least 9 weeks out of 16 weeks.

63. The method according to claim 56, wherein the sustained relief occurs for at least 6 weeks out of 16 weeks.

64. The method according to claim 56, wherein the sustained relief occurs for at least 3 weeks out of 4 weeks.

65. The method according to claim 56, wherein the sustained relief occurs for at least 1 week out of 2 weeks.

66. The method according to claim 56, wherein the sustained relief occurs for at least 1 week.

67. The method according to claim 56, wherein the sustained relief occurs for at least 2 weeks.

68. The method according to any one of claims 55-67, wherein the GC-C agonist is linaclotide.

69. The method according to any one of claims 1-68, wherein administering of linaclotide decreases abdominal pain, abdominal discomfort, or abdominal bloating in a patient with greater than moderate abdominal pain prior to the administration of linaclotide.

70. The method according to any one of claims 1-68, wherein administering of linaclotide decreases abdominal pain, abdominal discomfort, or abdominal bloating in a patient with moderate to severe abdominal pain.

71. The method according to any one of claims 1-68, wherein administering of linaclotide decreases abdominal pain, abdominal discomfort, or abdominal bloating in a patient with severe to very severe abdominal pain.

72. The method according to any one of claims 1-71, wherein administering of linaclotide improves a bowel symptom selected from:
an increase in the CSBMs/per week;
an increase in the SBMs/per week;
an increase in stool consistency; or
a decrease in straining during defecation;
in a patient with greater than moderate abdominal pain.

73. The method according to any one of claims 1-71, wherein administering of linaclotide improves a bowel symptom selected from:
an increase in the CSBMs/per week;
an increase in the SBMs/per week;
an increase in stool consistency; or
a decrease in straining during defecation;
in a patient with moderate to severe abdominal pain.

74. The method according to any one of claims 1-71, wherein administering of linaclotide improves a bowel symptom selected from:
an increase in the CSBMs/per week;
an increase in the SBMs/per week;
an increase in stool consistency; or
a decrease in straining during defecation;
in a patient with severe to very severe abdominal pain.

75. The method according to claim 56, wherein the sustained relief occurs for at least 26 weeks.

76. A method of treating a patient with constipation predominant irritable bowel syndrome, comprising administering a therapeutically effective dose of a GC-C agonist, wherein the GC-C agonist produces a rapid relief within one week and sustained relief for 26 weeks of symptoms to constipation predominant irritable bowel syndrome.
